(19) United States
(12) Patent Application Publication
(10) Pub. No.: US 2024/0100200 A1
(43) Pub. Date: Mar. 28, 2024

(54) COMPOSITIONS COMPRISING ASCORBIC ACID AND AN IMAGING AGENT AND RELATED METHODS

(71) Applicant: Lantheus Medical Imaging, Inc., North Billerica, MA (US)

(72) Inventors: James F. Castner, Groton, MA (US); Dianne D. Zdankiewicz, Londonderry, NH (US); James E. Anderson, Hudson, MA (US)

(73) Assignee: Lantheus Medical Imaging, Inc., North Billerica, MA (US)

(21) Appl. No.: 18/357,073

(22) Filed: Jul. 21, 2023

Related U.S. Application Data
(63) Continuation of application No. 16/228,691, filed on Dec. 20, 2018, now abandoned.
Application No. 16/228,691 is a continuation of application No. 14/352,742, filed on Jul. 18, 2014, now abandoned.

(51) Int. Cl. A61K 51/04; A61K 47/22

(52) U.S. Cl. CPC A61K 51/0459; A61K 47/22

(57) ABSTRACT

The present invention is generally directed towards compositions comprising ascorbic acid or ascorbate salt and an imaging agent, and related methods. In some embodiments, the imaging agent comprises pyridaben or a pyridaben analog attached to an imaging moiety.

### FIELD OF THE INVENTION

The present invention is generally directed towards compositions comprising ascorbic acid or ascorbate salt and an imaging agent, and related methods. In some embodiments, the imaging agent comprises pyridaben or a pyridaben analog attached to an imaging moiety.

### BACKGROUND OF THE INVENTION

Radiopharmaceuticals are radionuclide-containing compounds. Radiopharmaceuticals are routinely used in nuclear medicine for diagnosis (e.g., as an imaging agent) or therapy of various diseases. Decomposition of the radiopharmaceutical composition prior to administration can decrease the diagnostic and/or therapeutic efficacy and/or increase the toxicity of the radiopharmaceutical composition.

### SUMMARY OF THE INVENTION

In one aspect, a composition is provided comprising an imaging agent comprising pyridaben or a pyridaben analog attached to an imaging moiety; and ascorbic acid, wherein the pH of the composition between about 1.5 and 3.5 and wherein ascorbic acid is present at a concentration between about 20 mg/mL and about 200 mg/mL. It is to be understood that as used herein, the term “between” includes the outer limits of the specified range. As an example, a pH that is between 1.5 and 3.5, as used herein, means a pH that is 1.5, 3.5 or any pH therebetween.

In some embodiments, the pH of the composition is between about 1.5 and about 1.9. In some embodiments, the pH of the composition is between about 2.1 and about 3.5. In some embodiments, the pH of the composition is between about 2.5 and about 3.5. In some embodiments, the pH of the composition is between about 2.1 and about 2.3. In some embodiments, the pH of the composition is not 2. In some embodiments, the pH of the composition is not 2.4. In some embodiments, the pH of the composition is not between 1.6 and 2.4

In some embodiments, ascorbic acid is present in a concentration between about 20 mg/mL and about 49 mg/mL. In some embodiments, ascorbic acid is present in a concentration between about 51 mg/mL and about 200 mg/mL. In some embodiments, ascorbic acid is present in a concentration between about 21 mg/mL and about 49 mg/mL. In some embodiments, ascorbic acid is present in a concentration between about 51 mg/mL and about 199 mg/mL. In some embodiments, ascorbic acid is present in a concentration between about 51 mg/ml and about 99 mg/mL. In some embodiments, ascorbic acid is present in a concentration between about 101 mg/mL and about 199 mg/mL. In some embodiments, ascorbic acid concentration is 50 mg/mL. In some embodiments, ascorbic acid concentration is not 50 mg/mL In some embodiments, ascorbic acid concentration is not 20 mg/mL. In some embodiments, ascorbic acid concentration is not 100 mg/mL. In some embodiments, ascorbic acid concentration is not 200 mg/nL. In some embodiments, ascorbic acid concentration is not 0.28 M.

In some embodiments, the composition further comprises water. In some embodiments, the composition further comprises acetonitrile.

In some embodiments, the radioactive concentration of the composition is between about 1 mCi/mL and about 200 mCi/mL. In some embodiments, the radioactive concentration of the composition is less than or equal to about 65 mCi/mL.

In another aspect, a diagnostic composition is provided comprising an imaging agent comprising pyridaben or a pyridaben analog attached to an imaging moiety; and ascorbic acid, wherein the pH of the composition is between about 4.5 and 7.5, and wherein ascorbic acid is present in a concentration between about 20 mg/mL and about 200 mg/mL.

In some embodiments, the pH is between about 4.5 and about 5.7. In some embodiments, the pH is between about 5.9 and about 7.5. In some embodiments, the pH is between 4.6 and 5.7. In some embodiments, the pH is between 4.7 and about 5.7. In some embodiments, the pH is between 5.9 and about 7.5. In some embodiments, the pH is between about 6.1 and about 7.5. In some embodiments, the pH is between 5.9 and about 6.4. In some embodiments, the pH is between about 6.6 and about 7.5. In some embodiments, the pH is not 5.8. In some embodiments, the pH is not 4.5. In some embodiments, the pH is not 4.6. In son embodiments, the pH is not 5. In some embodiments, the pH is not 6.0. In some embodiments, the pH is not 6.5.

In some embodiments, ascorbic acid is present in a concentration between about 20 mg/nL and about 49 mg/nL. In some embodiments, ascorbic acid is present in a concentration between about 51 mg/mL and about 200 mg/mL. In some embodiments, ascorbic acid is present in a concentration between about 2.1 ng/m/L and about 49 mg/mL In some embodiments, ascorbic acid is present in a concentration between about 51 mg/nL and about 199 mg/mL. In some embodiments, ascorbic acid is present in a concentration between about 51 mg/ml and about 99 mg/mL. In some embodiments, ascorbic acid is present in a concentration between about 101 mg/mL and about 199 mg/mL. In some embodiments, ascorbic acid concentration is 50 mg/nL. In some embodiments, ascorbic acid concentration is not 50 mg/mL. In some embodiments, ascorbic acid concentration is not 20 mg/nL. In some embodiments, ascorbic acid concentration is not 100 mg/mL. In some embodiments, ascorbic acid concentration is not 200 mg/nL. In some embodiments, ascorbic acid concentration is not 0.28 M.

In some embodiments, the composition further comprises water. In some embodiments, the composition further comprises an alcohol. In some embodiments, the alcohol is ethanol. In some embodiments, ethanol is present in less than about 5% by volume. In some embodiments, ethanol is present in about 5% by volume, or about 4% by volume, or about 3% by volume, or about 2% by volume, or about 1% by volume.

In some embodiments, the radioactive concentration of the composition or the diagnostic composition is about 1 mCi/mL, about 2 mCi/mL, about 3 mCi/mL, about 4 mCi/mL about 5 mCi/mL, about 6 mCi/mL, about 7 mCi/mL, about 8 mCi/mL, about 9 mCi/mL, or about 10 mCi/mL. In some embodiments, the radioactive concentration of the composition or the diagnostic composition is between about 1 mCi/mL and about 200 mCi/mL. In some embodiments, the radioactive concentration of the composition or the diagnostic composition is between about 2 mCi/mL and about 160 mCi/mL, or between about 2 mCi/mL and about 150 mCi/mL, or between about 5 mCi/mL and about 140 mCi/mL, or between about 10 mCi/mL and about 130 mCi/mL, or between about 10 mCi/mL and about 120 mCi/mL, or between about 10 mCi/mL and about 110 mCi/mL, or between about 20 mCi/mL and about 100 mCi/mL, or between about 30 mCi/mL and about 100 mCi/mL, or between about 40 mCi/mL and about 100 mCi/mL, between about 30 mCi/mL and about 120 mCi/mL, or between about 40 mCi/mL and about 120 mCi/mL, or between about 50 mCi/mL and about 100 mCi/mL, or between about 30 mCi/ML and about 90 mCi/mL, or between about 40 mCi/mL and about 80 mCi/mL, or between about 50 mCi/mL and about 70 mCi/mL.

In some embodiments, the composition has a radiochemical purity of at least about 95%. In some embodiments, the composition has a radiochemical purity between about 95% and about 99%. In some embodiments, the composition has a radiochemical purity of at least 95% for at least 12 hours. In some embodiments, the composition has a radiochemical purity of 95% to 98% for at least 12 hours. In some embodiments, the composition has a radiochemical purity of at least 99% for at least 12 hours.

For the aspects described above, in some embodiments, the imaging agent has a structure as in formula (I),

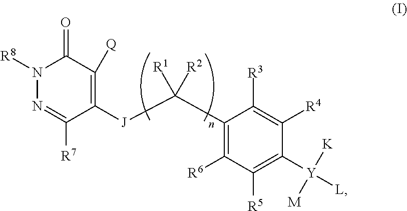

wherein:
J is selected from N(R⁹), S, O, C(═O), C(═O)O, NHCH₂CH₂O, a bond, or C(═O)N(R⁷);
when present, K is selected from hydrogen, alkoxyalkyl, alkyloxy, aryl, C₁-C₆ alkyl, heteroaryl, and an imaging moiety;
when present, L is selected from hydrogen, alkoxyalkyl, alkyloxy, aryl, C₁-C₆ alkyl, heteroaryl, and an imaging moiety;
M is selected from hydrogen, alkoxyalkyl, alkyloxy, aryl, C₁-C₆ alkyl, heteroaryl, and an imaging moiety; or
L and M, together with the atom to which they are attached, form a three-, four-, five-, or six-membered carbocyclic ring;
Q is halo or haloalkyl;
n is 0, 1, 2, or 3;
R¹, R², R⁷, and R⁹ are independently selected from hydrogen, C₁-C₆ alkyl, and an imaging moiety;
R³, R⁴, R⁵, and R⁶ are independently selected from hydrogen, halogen, hydroxyl, alkyloxy, C₁-C₆ alkyl, and an imaging moiety;
R⁸ is C₁-C₆ alkyl; and
Y is selected from a bond, carbon, and oxygen; provided that when Y is a bond, K and L are absent and M is selected from aryl and heteroaryl; and provided that when Y is oxygen, K and L, are absent and M is selected from hydrogen, alkoxyalkyl, aryl, C₁-C₆ alkyl, and heteroaryl;
wherein each occurrence of alkoxyalkyl, alkyloxy, aryl, C₁-C₆ alkyl, and heteroaryl is optionally substituted with an imaging moiety, provided that at least one imaging moiety is present in formula (I).

In some embodiments, J is O; M is selected from alkoxyalkyl, alkyloxy, aryl, C₁-C₆ alkyl, and heteroaryl, each optionally substituted with an imaging moiety; Q is halo or haloalkyl; n is 1; and R⁸ is C₁-C₆ alkyl.

In some embodiments, J is O; M is alkyloxy substituted with an imaging moiety; Q is halo; n is 1; and R⁸ is C₁-C₆ alkyl.

In some embodiments, J is O; and R⁸ is tert-butyl. In some embodiments, Q is halo. In some embodiments. Q is chloro. In some embodiments, M is alkyloxy substituted with an imaging moiety.

In some embodiments, the imaging moiety is a radioisotope for nuclear medicine imaging, a paramagnetic species for use in MRI imaging, an echogenic entity for use in ultrasound imaging, a fluorescent entity for use in fluorescence imaging, or a light-active entity for use in optical imaging. In some embodiments, the paramagnetic species for use in MRI imaging is Gd³⁺, Fe³⁺, In³⁺, or Mn²⁺. In some embodiments, the echogenic entity for use in ultrasound imaging is a surfactant encapsulated fluorocarbon microsphere. In some embodiments, the radioisotope for nuclear medicine imaging is ¹¹C, ¹³N, ¹⁸F, ¹²³I, ¹²⁵I, ^{99m}Tc, ⁹⁵Tc, ¹¹¹In, ⁶Cu, ⁶⁴Cu, ⁶⁷Ga, or ⁶⁸Ga. In some embodiments, the imaging moiety is ¹⁸F.

In some embodiments, the imaging agent is selected from the group consisting of

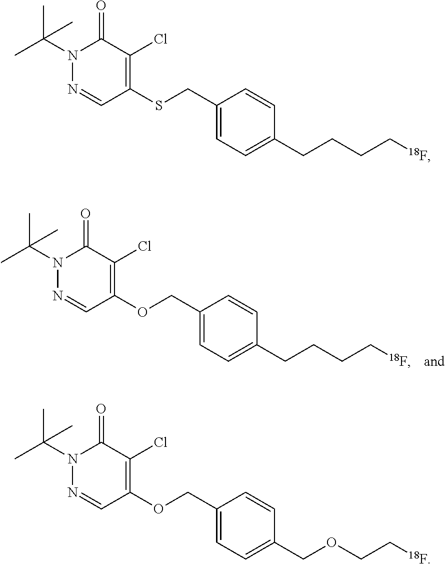

In one embodiments, a composition is provided comprising ascorbic acid and an imaging agent, wherein the imaging agent comprises pyridaben or a pyridaben analog attached to an imaging moiety, including a radioactive imaging moiety such as ¹⁸F, wherein the pH of the composition is between about 4.5 and 7.5, and wherein ascorbic acid is present in a concentration between about 20 mg/mL and about 200 mg/mL, and wherein radiochemical purity is at least about 95%, at least about 96%, at least about 97%, at least about 98%, at least about 98.5%, at least about 98.9%, at least about 99%, at least about 99.5%, at least about 99.9%. The ascorbic acid concentration may be about 50 mg/mL. The pH may be about 5.8. The total amount of radioactivity in the composition may be about 3 mCi, about 6.5 mCi, about 9.5 mCi, or about 12.5 mCi, and optionally the volume may be equal to or less than about 6 mL. The imaging agent may be, but is not limited to, any of the three foregoing ¹⁸F-labeled imaging agents.

In some embodiments, a composition is provided comprising ascorbic acid and an imaging agent, wherein the imaging agent comprises pyridaben or a pyridaben analog attached to an imaging moiety, including a radioactive imaging moiety such as ¹⁸F, wherein the pH of the composition is between about 4.5 and 7.5, and wherein ascorbic acid is present in a concentration between about 20 mg/mL and about 200 mg/mL, and wherein the radiochemical is between about 95% and about 98%, between about 95% and about 98.5%, between about 95% and about 98.9%, between about 95% and about 99%, between about 95% and about 99.5%, between about 95% and about 99.9%, or between about 95% and about 100%. The ascorbic acid concentration may be about 50 mg/mL. The pH may be about 5.8. The ascorbic acid concentration may be about 50 mg/mL and the pH may be about 5.8. The total amount of radioactivity in the composition may be about 3 mCi, about 6.5 mCi, about 9.5 mCi, or about 12.5 mCi, and optionally the volume may be equal to or less than about 6 mL. The imaging agent may be, but is not limited to, any of the three foregoing ¹⁸F-labeled imaging agents.

In yet another aspect, methods are provided comprising administering a composition to a subject and obtaining an image of the subject. In some embodiments, the subject is a human subject. In some embodiments, the image is an image of a cardiovascular region of the subject. In some embodiments, the composition is a diagnostic composition.

In still yet another aspect, use of the composition described herein is provided for obtaining an image of a subject. In some embodiments, the subject is a human subject. In some embodiments, the image is an image of a cardiovascular region of the subject. In some embodiments, the composition is a diagnostic composition.

### BRIEF DESCRIPTION OF THE DRAWINGS

FIG. 1 shows a plot of radiochemical purity of 2-tert-butyl-4-chloro-5-[4-(2-[18F]fluoro-ethoxymethyl)-benzyloxy]-2H-pyridazin-3-one as a function of time in compositions having varying pH levels.

FIG. 2 shows a plot of the rate of impurity formation for various 2-tert-butyl-4-chloro-5-[4-(2-fluoro-ethoxymethyl)-benzyloxy]-2H-pyridazin-3-one compositions at a pH of (a) 4.0, (b) 8.2, (c) 6.3, (d), 5.4, (e) 6.0, or (f) 4.5.

FIG. 3 shows a plot of radiochemical purity of 2-tert-butyl-4-chloro-5-[4-(2-[18F]fluoro-ethoxymethyl)-benzyloxy]0.2H-pyridazin-3-one in a series of solutions comprising ascorbic acid at a concentration of (a) 20 mg/mL (|p|>0.001), (b) 50 mg/mL, (c) 100 mg/mL, (d) and 200 mg/mL.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION OF THE INVENTION

The present invention generally relates to compositions comprising ascorbic acid or ascorbate salts and an imaging agent, and related methods. In some embodiments, the imaging agent comprises pyridaben or a pyridaben analog attached to an imaging moiety. As discussed in greater detail herein, imaging agents may be attached to imaging moieties that are radionuclides (or radioisotopes), and accordingly such imaging agents may be referred to herein as radiopharmaceuticals.

In one aspect of the invention, a composition is provided comprising ascorbic acid and an imaging agent, wherein the imaging agent comprises pyridaben or a pyridaben analog attached to an imaging moiety, wherein the pH of the composition is between about 1.5 and 3.5, and wherein ascorbic acid is present in a concentration between about 20 mg/mL and about 200 mg/mL.

In this and other aspects and embodiments of the invention, ascorbic acid may be present in an acidic form (e.g., as ascorbic acid) and/or basic form (e.g., as ascorbate), depending on pH. For example, at pH values greater than about 4.2 (i.e., the pKa of ascorbic acid), the basic form will be more prevalent than the acidic form. The higher the pH, the higher the proportion that is present as the basic form. Conversely, at pH values less than about 4.2, the acidic form will be more prevalent than the basic form. The lower the pH, the higher the proportion that is present as the acidic form. Accordingly, where the term ascorbic acid is used herein in connection with a composition, it should be understood that the composition may comprise the acidic form of ascorbic acid, the basic form of ascorbic acid, or combinations thereof.

The basic form (i.e., ascorbate) may be associated with a counter ion. Those of ordinary skill in the art will be aware of pharmaceutically acceptable salts suitable for association with ascorbate and for use with the compositions described herein. Non-limiting examples of pharmaceutically acceptable salts are described herein. In some cases, the counter ion is sodium (e.g., such that the composition comprises sodium ascorbate).

In some embodiments, the pH of the composition is about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, or about 3.5. In some embodiments, the pH of the composition is between about 1.5 and less than about 3.5. In some embodiments, the pH of the composition is between about 1.5 and about 3.0. In some embodiments, the pH of the composition is between about 1.5 and about 2.5. In some embodiments, the pH of the composition is between about 1.5 and about 1.9. In some embodiments, the pH of the composition is between about 1.5 and about 1.6. In some embodiments, the pH of the composition is between about 2.1 and about 3.5. In some embodiments, the pH of the composition is between about 2.4 and about 3.5. In some embodiments, the pH of the composition is between about 2.5 and about 3.5. In some embodiments, the pH of the composition is between about 2.1 and about 2.3.

In some embodiments, the pH of the composition is not 2. In some embodiments, the pH of the composition is not 2.4. In some embodiments, the pH of the composition is not between 1.6 and 2.4.

In some embodiments, ascorbic acid is present in a concentration that is about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, ascorbic acid is present in a concentration between about 30 mg/mL and about 200 mg/mL. In some embodiments, ascorbic acid is present in a concentration between about 40 mg/mL and about 200 mg/mL. In some embodiments, ascorbic acid is present in a concentration between about 50 mg/mL and about 200 mg/mL. In some embodiments, ascorbic acid is present in a concentration between about 75 mg/mL and about 200 mg/mL. In some embodiments, ascorbic acid is present in a concentration between about 100 mg/mL and about 200 mg/mL. In some embodiments, ascorbic acid is present in a concentration between about 110 mg/mL and about 200 mg/mL. In some embodiments, ascorbic acid is present in a concentration between about 20 mg/mL and about 49 mg/mL. In some embodiments, ascorbic acid is present in a concentration between about 21 mg/mL and about 49 mg/mL. In some embodiments, ascorbic acid is present in a concentration between about 51 mg/mL and about 200 mg/mL. In some embodiments, ascorbic acid is present in a concentration between about 51 mg/mL and about 199 mg/mL. In some embodiments, ascorbic acid is present in a concentration between about 51 mg/mL and about 99 mg/mL. In some embodiments, ascorbic acid is present in a concentration between about 101 mg/mL and about 199 mg/mL.

In some embodiments, the ascorbic acid concentration is not 20 mg/mL In some embodiments, the ascorbic acid concentration is not 50 mg/mL. In some embodiments, the ascorbic acid concentration is not 100 mg/mL. In some embodiments, the ascorbic acid concentration is not 200 mg/mL. In some embodiments, the ascorbic acid concentration is not 0.28 M.

In one embodiment, the pH of the composition is not 2 and the concentration of ascorbic acid is not 0.28 M. In another embodiment, the pH of the composition is not between 1.6 and 2.4 and the concentration of the ascorbic acid is not 0.28 M. In yet another embodiment, the pH of the composition is not 2 and the concentration of ascorbic acid is not 50 mg/mL or not less than 50 mg/mL. In another embodiment, the pH of the composition is not between 1.6 and 2.4 and the concentration of the ascorbic acid is not 50 mg/mL or not less than 50 mg/nL.

In some embodiments, the composition further comprises at least one solvent. The imaging agent and/or the ascorbic acid may be substantially soluble in the solvent. In some cases, the composition comprises water. In some cases, the composition comprises water and at least one additional solvent, wherein the solvent may be substantially miscible with the water. Non-limiting examples of solvents include, but are not limited to, ether solvents (e.g., tetrahydrofuran, and dimethoxyethane), and alcohol solvents (e.g., ethanol, methanol, propanol, isopropanol, tert-butanol). Other non-limiting examples of solvents include acetone, acetic acid, formic acid, dimethyl sulfoxide, dimethyl formamide, acetonitrile, glycol, triethylamine, picoline, and pyridine. In some embodiments, the composition comprises water and a polar solvent substantially miscible with the water.

In some embodiments, the composition comprises water and acetonitrile. In some cases, the acetonitrile is present in between about 5% and about 60% by volume, or between about 10% and about 60% by volume, or between about 20% and about 60% by volume, or between about 30% and about 60% by volume, or between about 40% and about 60% by volume, or between about 50% and about 60% by volume, or between about 5% and about 50% by volume, or between about 5% and about 40% by volume, or between about 5% and about 30% by volume, or between about 5% and about 25% by volume. In some cases, the acetonitrile is present in about 5% by volume, about 10% by volume, about 15% by volume, about 20% by volume, about 25% by volume, about 30% by volume, about 40% by volume, about 50% by volume, or about 60% by volume. In some cases, the acetonitrile is present in greater than about 5% by volume. In some cases, the acetonitrile is present in less than about 60% by volume.

In another aspect of the invention, a composition is provided comprising ascorbic acid and an imaging agent, wherein the imaging agent comprises pyridaben or a pyridaben analog attached to an imaging moiety, wherein the pH of the composition is between about 4.5 and 7.5, and wherein ascorbic acid is present in a concentration between about 20 mg/mL and about 200 mg/mL.

In some embodiments, the composition is a diagnostic composition. The term “diagnostic composition” refers to a composition for use in diagnostic applications, preferably in human subjects. The composition may be used to diagnose a condition, disorder, or disease, as described in greater detail herein. The composition typically will be administered to a subject, such as a human subject, and thus should be suitable for in vivo use. In some cases, the radioactive concentration of the composition is about 1 mCi/mL, about 2 mCi/mL, about 3 mCi/mL, about 4 mCi/mL, about 5 mCi/mL, about 6 mCi/mL, about 7 mCi/mL, about 8 mCi/mL, about 9 mCi/mL, or about 10 mCi/mL.

In some embodiments, the pH of the composition is about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the p of the composition is between greater than 6 and about 7.5. In some embodiments, the pH of the composition is between about 4.5 and about 5.7. In some embodiments, the pH of the composition is between about 4.6 and about 5.7. In some embodiments, the pH of the composition is between about 4.7 and about 5.7. In some embodiments, the pH of the composition is between about 5.9 and about 7.5. In some embodiments, the pH of the composition is between about 6.1 and about 7.5. In some embodiments, the pH of the composition is between about 5.9 and about 6.4. In some embodiments, the pH of the composition is between about 6.6 and about 7.5.

In some embodiments, the pH of the composition is not 4.5. In some embodiments, the pH of the composition is not 4.6. In some embodiments, the pH of the composition is not 5.8. In some embodiments, the pH of the composition is not 6.0. In some embodiments, the pH of the composition is not 6.5.

In some embodiments, ascorbic acid is present in a concentration that is about 20 mg/mL, about 30 mg/mL, about 40 mg/mL, about 50 mg/mL, about 60 mg/nL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, ascorbic acid is present in a concentration between about 30 mg/mL and about 200 mg/mL. In some embodiments, ascorbic acid is present in a concentration between about 40 mg/mL and about 200 mg/mL. In some embodiments, ascorbic acid is present in a concentration between about 50 mg/mL and about 200 mg/mL. In some embodiments, ascorbic acid is present in a concentration between about 75 mg/mil and about 200 mg/mL. In some embodiments, ascorbic acid is present in a concentration between about 100 mg/mL and about 200 mg/mL. In some embodiments, ascorbic acid is present in a concentration between about 110 mg/mL and about 200 mg/mL. In some embodiments, ascorbic acid is present in a concentration between about 20 mg/mL and about 49 mg/mL. In some embodiments, ascorbic acid is present in a concentration between about 21 mg/nL and about 49 mg/mL. In some embodiments, ascorbic acid is present in a concentration between about 51 mg/mL and about 200 mg/mL. In some embodiments, ascorbic acid is present in a concentration between about 51 mg/mL and about 199 mg/mL. In some embodiments, ascorbic acid is present in a concentration between about 51 mg/mL and about 99 mg/mL. In some embodiments, ascorbic acid is present in a concentration between about 101 mg/mL and about 199 mg/mL.

In some embodiments, the ascorbic acid concentration is not 20 mg/mL. In some embodiments, the ascorbic acid concentration is not 50 mg/mL. In some embodiments, the ascorbic acid concentration is not 100 mg/mL. In some embodiments, the ascorbic acid concentration is not 200 mg/mL. In some embodiments, the ascorbic acid concentration is not 0.28 M.

In one embodiment, the pH of the composition is not 5.8 and the concentration of ascorbic acid is not 0.28M. In one embodiment, the pH of the composition is not 5.8 and the concentration of ascorbic acid is not 50 mg/L or not less than 50 mg/mL.

In some embodiments, the composition further comprises at least one solvent. The imaging agent and/or the ascorbic acid may be substantially soluble in the solvent. In some cases, the composition comprises water. In some cases, the composition comprises water and at least one additional solvent, wherein the solvent may be substantially miscible with the water. Non-limiting examples of solvents include, but are not limited to, ether solvents (e.g., tetrahydrofuran, and dimethoxyethane), and alcohol solvents (e.g., ethanol, methanol, propanol, isopropanol, tert-butanol). Other non-limiting examples of solvents include acetone, acetic acid, formic acid, dimethyl sulfoxide, dimethyl formamide, acetonitrile, glycol, triethylamine, picoline, and pyridine. In some embodiments, the composition comprises water and a polar solvent substantially miscible with the water.

In some embodiments, the composition further comprises water and an alcohol. In some cases, the composition comprises water and a pharmaceutically acceptable alcohol. Non-limiting examples of pharmaceutically acceptable alcohols include ethanol, propanol (e.g., isopropanol) propylene glycol, benzyl alcohol, and glycerol. The alcohol may be present in less than about 10% by volume, 9% by volume, 8% by volume, 7% by volume, 6% by volume, 5% by volume, 4% by volume, 3% by volume, 2% by volume, or 1% by volume. In some embodiments, the alcohol is present in about 5% by volume or in less than about 5% by volume. In some cases, the alcohol is present in between about 0.1% and about 5% by volume.

In some embodiments, the composition comprises water and ethanol. In some cases, the ethanol is present in less than about 5% by volume. In some cases, the ethanol is present in about 5% by volume, about 4% by volume, about 3% by volume, about 2% by volume, or about 1% by volume. In some cases, the ethanol is present in between about 0.1% and about 5% by volume.

In some embodiments, a diagnostic composition of the invention may be produced by a method comprising the steps of:
a) providing a first solution comprising the imaging agent and ascorbic acid, wherein the first solution has a pH between about 1.5 and about 3.5 and wherein ascorbic acid is present in a concentration between about 20 mg/mL and about 200 mg/mL;
b) applying the first solution to a resin and washing the resin with a second solution, wherein the imaging agent is substantially retained on the resin during the washing, wherein the second solution has a pH between about 1.5 and about 3.5 and wherein ascorbic acid is present in a concentration between about 20 mg/ml and about 200 mg/mL;
c) eluting the imaging agent from the resin with an eluting solution comprising an alcohol to form a third solution comprising the alcohol and the imaging agent; and
d) diluting the third solution with a fourth solution comprising ascorbic acid, wherein the fourth solution has a pH between about 4.5 to about 7.5 and has ascorbic acid present in a concentration between about 20 mg/ml and about 200 ng/ml, thereby forming the diagnostic composition.

Without wishing to be bound by theory, this exemplary method may be useful to remove impurities from a composition comprising the imaging agent and/or to exchange the solvent in which the imaging agent is present, thus allowing for formation of a diagnostic composition. For example, the first solution may be obtained from the synthesis of the imaging agent (e.g., via HPLC or another purification method), and may comprise impurities and/or solvents which are not suitable for administration to a subject. Accordingly, the impurities may be removed and/or the solvents may be exchanged using a method as described above.

For example, the first solution may comprise ascorbic acid, the imaging agent, and one or more solvents and/or impurities. The first solution may be applied to a resin, wherein the imaging agent is substantially retained on the resin and the other components (e.g., solvents such as acetonitrile and/or impurities) may be removed via elution (e.g., in step b, by washing the resin with the second solution). The imaging agent may be recovered from the resin by eluting the imaging agent with the third solvent (e.g., step c). The resulting solution may then be further diluted, if desired, to form a diagnostic composition suitable for administration to a subject (e.g., step d).

In one embodiment, the first solution comprises water and acetonitrile (or another solvent, for example, which is not suitable for administration to a subject). The water and the acetonitrile (and/or impurities) may not adhere to the resin and may thus be eluted. Accordingly, the third solution formed by eluting the imaging agent from the resin may not comprise the acetonitrile (or other solvent). In some cases, the first solution may be a composition according to the first aspect of the invention described herein. Non-limiting examples of solvents include, but are not limited to, ether solvents (e.g., tetrahydrofuran, and dimethoxyethane), and alcohol solvents (e.g., ethanol, methanol, propanol, isopropanol, tert-butanol). Other non-limiting examples of solvents include acetone, acetic acid, formic acid, dimethyl sulfoxide, dimethyl formamide, acetonitrile, glycol, triethylamine, picoline, and pyridine. In some embodiments, the composition comprises water and a polar solvent substantially miscible with the water.

In some cases, the first solution comprises water and acetonitrile. In some cases, the acetonitrile is present in between about 5% and about 60% by volume, or between about 10% and about 60% by volume, or between about 20% and about 60% by volume, or between about 30% and about 60% by volume, or between about 40% and about 60% by volume, or between about 50% and about 60% by volume, or between about 5% and about 50% by volume, or between about 5% and about 40% by volume, or between about 5% and about 30% by volume, or between about 5% and about 25% by volume. In some cases, the acetonitrile is present in about 5% by volume, about 10% by volume, about 15% by volume, about 20% by volume, about 25% by volume, about 30% by volume, about 40% by volume, about 50% by volume, or about 60% by volume. In some cases, the acetonitrile is present in greater than about 5% by volume. In some cases, the acetonitrile is present in less than about 60% by volume.

The composition of the fourth solution generally depends on the desired formulation of the final diagnostic composition. That is, the components of the fourth solution may be chosen such that combination of the third solution and the fourth solution results in the final diagnostic composition. In some cases, the third solution comprising the imaging agent and an alcohol is diluted with a selected fourth solution so that the final diagnostic composition with the desired concentrations and conditions (e.g., pH) is obtained. For example, if the third solution comprises the imaging agent and neat or essentially neat alcohol (e.g., ethanol) and the final diagnostic composition is to comprise less than 5% ethanol by volume, the third solution may be diluted by at least a factor of at least about 20 with the fourth solution (e.g., having the pH and concentration of ascorbic acid desired for the final formulation).

The eluting solvent may be any solvent which allows for elution of the imaging agent. Generally, the imaging agent is substantially soluble in the eluting solvent. In some cases, the solvent in the eluting solution is an alcohol. For example, the alcohol may be the alcohol contained in the final diagnostic composition. For example, as described above, in some embodiments, the alcohol may be a pharmaceutically acceptable alcohol. In some cases, the alcohol is ethanol. The alcohol may be neat and/or may comprise water. Generally, the solution comprises at least 50% alcohol, at least 60% alcohol, at least 70% alcohol, at least 80% alcohol, at least 80% alcohol, at least 90% alcohol, at least 95% alcohol, at least 97% alcohol, at least 98% alcohol, at least 99% alcohol, at least 99.5% alcohol, or more.

In some cases, the third solution is diluted with the fourth solution by addition of the third solution to the fourth solution. For example, a syringe ray be provided comprising the fourth solution, and the third solution may be drawn into the syringe, thus adding the third solution to the fourth solution. In other cases, the third solution may be diluted with the fourth solution by addition of the fourth solution to the third solution.

In some embodiments, the pH of the first solution, the second solution, and/or the third solution is about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, or about 3.5. In some embodiments, the pH of the first solution, the second solution, and/or the third solution is between about 1.5 and about 1.6. In some embodiments, the pH of the first solution, the second solution, and/or the third solution is between about 1.5 and about 1.9. In some embodiments, the pH of the first solution, the second solution, and/or the third solution is between about 2.1 and about 3.5. In some first solution, the second solution, and/or the third solution, the pH of the first solution, the second solution, and/or the third solution is between about 2.4 and about 3.5. In some embodiments, the pH of the first solution, the second solution, and/or the third solution is between about 2.5 and about 3.5. In some embodiments, the pH of the first solution, the second solution, and/or the third solution is between 2.1 and about 2.3. In some embodiments, the pH of the first solution, the second solution, and/or the third solution is not 2. In some embodiments, the pH of the first solution, the second solution, and/or the third solution is not 2.4. In some embodiments, the pH of the first solution, the second solution, and/or the third solution is not between about 1.6 and about 2.4. The pHs of the first, second, and third solutions may be the same or they may be different.

In some embodiments, in the first solution, the second solution, the third solution, and/or the fourth solution ascorbic acid is present in a concentration that is about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, in the first solution, the second solution, the third solution, and/or the fourth solution ascorbic acid is present in a concentration between about 20 mg/mL and about 49 mg/mL. In some embodiments, in the first solution, the second solution, the third solution, and/or the fourth solution ascorbic acid is present in a concentration between about 21 mg/n/L and about 49 mg/mL. In some embodiments, in the first solution, the second solution, the third solution, and/or the fourth solution ascorbic acid is present in a concentration between about 51 mg/mL and about 200 mg/mL. In some embodiments, in the first solution, the second solution, the third solution, and/or the fourth solution ascorbic acid is present in a concentration between about 51 mg/mL and about 199 mg/mL. In some embodiments, in the first solution, the second solution, the third solution, and/or the fourth solution ascorbic acid is present in a concentration between about 51 mg/mL and about 99 mg/mL. In some embodiments, in the first solution, the second solution, the third solution, and/or the fourth solution ascorbic acid is present in a concentration between about 101 mg/nL and about 199 mg/mL. The ascorbic acid concentrations in the first, second, third and fourth solutions may be the same or they may be different.

In some embodiments, in the first solution, the second solution, the third solution, and/or the fourth solution the ascorbic acid concentration is not 20 mg/mL. In some embodiments, in the first solution, the second solution, the third solution, and/or the fourth solution the ascorbic acid concentration is not 50 mg/mL. In some embodiments, in the first solution, the second solution, the third solution, and/or the fourth solution the ascorbic acid concentration is not 100 mg/mL. In some embodiments, in the first solution, the second solution, the third solution, and/or the fourth solution the ascorbic acid concentration is not 200 mg/mL. In some embodiments, the ascorbic acid concentration in the first solution, the second solution, the third solution, and/or the fourth solution is not 0.28 M.

In one embodiment, the pH of the first solution, the second solution, and/or the third solution is not 2 and the concentration of ascorbic acid is not 0.28M. In another embodiment, the pH of the first solution, the second solution, and/or the third solution is not between 1.6 and 2.4 and the concentration of the ascorbic acid is not 0.28M.

In one embodiment, the pH of the fourth solution is not 5.8 and the concentration of ascorbic acid is not 0.28M. In one embodiment, the pH of the fourth solution is not 5.8 and the concentration of ascorbic acid is not 50 mg/mL or not less than 50 mg/mL.

Suitable resins will be known to those of ordinary skill in the art. In one embodiment, the resin is a modified polymer. In another embodiment, the resin is a modified silica gel. In some embodiments, the silica gel is modified to be lipophilic. In some embodiments, the silica gel is modified with an alkyl chain. In a particular embodiment the resin is a C-18 resin.

### Radiochemical Purity, Stability, and Radioactive Concentration

The compositions described herein and/or prepared according to the methods described herein may have a high radiochemical purity and may maintain the high radiochemical purity for a substantial period of time.

As used herein, radiochemical purity refers to the proportion of the amount of radioactivity (from a given radioisotope) present in a specific radiopharmaceutical relative to the total amount of radioactivity (from the same radioisotope) in a composition that comprises the specific radiopharmaceutical. Radiochemical purity can be a measure of the degree of degradation and/or decomposition and/or conversion of the specific radiopharmaceutical into other compounds that may or may not comprise the radioisotope.

In some embodiments, a composition has a radiochemical purity of at least about 95%. In some embodiments, a composition has a radiochemical purity of at least about 96%. In some embodiments, a composition has a radiochemical purity of at least about 97%. In some embodiments, a composition has a radiochemical purity of at least about 98%. In some embodiments, a composition has a radiochemical purity of at least about 98.5%. In some embodiments, a composition has a radiochemical purity of at least about 98.9%. In some embodiments, a composition has a radiochemical purity of at least about 99%. In some embodiments, a composition has a radiochemical purity of at least about 99.5%. In some embodiments, a composition has a radiochemical purity of at least about 99.9%. In some embodiments, a composition has a radiochemical purity between about 95% and about 98%. In some embodiments, a composition has a radiochemical purity between about 95% and about 98.5%. In some embodiments, a composition has a radiochemical purity between about 95% and about 98.9%. In some embodiments, a composition has a radiochemical purity between about 95% and about 99%. In some embodiments, a composition has a radiochemical purity between about 95% and about 99.5%. In some embodiments, a composition has a radiochemical purity between about 95% and about 99.9%. In some embodiments, a composition has a radiochemical purity between about 95% and about 100%.

Those of ordinary skill in the art will be aware of techniques and systems for determining the radiochemical purity of a composition. In some cases, the radiochemical purity is determined using an HPLC associated with a radio-detector. Generally, the radiochemical purity is determined under ambient conditions (e.g., ambient temperature, ambient humidity, ambient light, etc.).

In some embodiments, a composition maintains a high radiochemical purity for a substantial period of time. Without wishing to be bound by theory, this may be due to the selection of appropriate composition components and conditions which aid in the stability of the imaging agent. For example, the presence of ascorbic acid and/or selection of an appropriate composition pH can greatly affect the radiostability of the imaging agent.

In some embodiments, a composition has a radiochemical purity of at least about 95% over a period of at least about 6 hours, at least 8 hours, at least 12 hours, at least 14 hours, or at least 16 hours. In some embodiments, a composition has a radiochemical purity of at least about 95% at about 12 hours. In some embodiments, a composition has a radiochemical purity of at least about, 97% at about 12 hours. In some embodiments, a composition has a radiochemical purity of at least 99% for at least 12 hours.

In some cases, the radioactive concentration of the composition is between about 1 mCi/mL and about 200 mCi/mL, between about 2 mCi/mL and about 160 mCi/mL, or between about 2 mCi/mL and about 150 mCi/mL, or between about 5 mCi/mL and about 140 mCi/mL, or between about 10 mCi/mL and about 130 mCi/mL, or between about 10 mCi/mL and about 120 mCi/mL, or between about 10 mCi/mL and about 110 mCi/mL, or between about 20 mCi/mL and about 100 mCi/nL, or between about 30 mCi/mL and about 100 mCi/mL, or between about 40 mCi/mL and about 100 mCi/mL, between about mCi/mL and about 120 mCi/mL, or between about 40 mCi/mL and about 120 mCi/mL, or between about 50 mCi/mL and about 100 mCi/mL, or between about 30 mCi/mL and about 90 mCi/mL, or between about 40 mCi/mL and about 80 mCi/mL, or between about 50 mCi/mL and about 70 mCi/mL. In some embodiment, the radioactive concentration of the composition is less than or equal to about 65 mCi/mL. In a particular embodiment, the radioactive concentration of the composition is about 65 mCi/mL. In some cases, the radioactive concentration of the composition is about 1 mCi/mL, about 2 mCi/mL, about 3 mCi/mL, about 4 mCi/mL, about 5 mCi/mL, about 6 mCi/mL, about 7 mCi/mL, about 8 mCi/mL, about 9 mCi/mL, about 10 mCi/mL, about mCi/mL, about 20 mCi/mL, about 40 mCi/mL, about 50 mCi/mL, about 60 mCi/mL, about 65 mCi/mL, about 70 mCi/mL, about 80 mCi/mL, about 90 MCi/ML, about 1.00 mCi/mL, about 110 mCi/mL, about 120 mCi/mL, about 130 mCi/mL, about 140 mCi/mL about 150 mCi/mL, or about 160 mCi/mL.

In some embodiments, the total amount of radioactivity in the composition ranges from about 1 to about 50 mCi, about 1 to about 20 mCi, about 1 to about 10 mCi, or about 1 to about 5 mCi. In some embodiments, the total amount of radioactivity in the composition is about 3 mCi, and optionally the composition is provided in a syringe. In some embodiments, the total amount of radioactivity in the composition is about 6 or about 6.5 mCi, and optionally the composition is provided in a syringe. In some embodiments, the total amount of radioactivity in the composition is about 9 or about 9.5 mCi, and optionally the composition is provided in a syringe. In some embodiments, the total amount of radioactivity in the composition is about 12.5 mCi, and optionally the composition is provided in a syringe. In some embodiments, the composition has a volume equal to or less than about 6 mL.

In some embodiments, a composition is provided comprising ascorbic acid and an imaging agent, wherein the imaging agent comprises pyridaben or a pyridaben analog attached to an imaging moiety, including a radioactive imaging moiety such as ¹⁸F, wherein the pH of the composition is between about 4.5 and 7.5, and wherein ascorbic acid is present in a concentration between about 20 mg/mL and about 200 mg/mL, and wherein radiochemical purity is at least about 95%, at least about 96%, at least about 97%, at least about 98%, at least about 98.5%, at least about 98.9%, at least about 99%, at least about 99.5%, at least about 99.9%. The ascorbic acid concentration may be about 50 mg/mL. The pH may be about 5.8. The ascorbic acid concentration may be about 50 mg/mL and the pH may be about 5.8. The total amount of radioactivity in the composition may be about 3 mCi, about 6.5 mCi, about 9.5 mCi, or about 12.5 mCi, and optionally the volume may be equal to or less than about 6 mL.

In some embodiments, a composition is provided comprising ascorbic acid and an imaging agent, wherein the imaging agent comprises pyridaben or a pyridaben analog attached to an imaging moiety, including a radioactive imaging moiety such as F, wherein the pH of the composition is between about 4.5 and 7.5, and wherein ascorbic acid is present in a concentration between about 20 mg/mL and about 200 mg/mL, and wherein the radiochemical is between about 95% and about 98%, between about 95% and about 98.5%, between about 95% and about 98.9%, between about 95% and about 99%, between about 95% and about 99.5%, between about 95% and about 99.9%, or between about 95% and about 100%. The ascorbic acid concentration may be about 50 mg/mL. The pH may be about 5.8. The ascorbic acid concentration may be about 50 mg/mL and the pH may be about 5.8. The total amount of radioactivity in the composition may be about 3 mCi, about 6.5 mCi, about 9.5 mCi, or about 12.5 mCi, and optionally the volume may be equal to or less than about 6 mL.

In some embodiments, the foregoing compositions may be a diagnostic composition. In some embodiments, the radioactive concentration of the foregoing compositions is about 1 mCi/mL, about 2 mCi/mL, about 3 mCi/mL, about 4 mCi/mL, about 5 mCi/mL, about 6 mCi/mL, about 7 mCi/mL, about 8 mCi/mL, about 9 mCi/mL, or about 10 mCi/mL. In some cases, the pH of the composition is about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some cases, the pH of the composition is between greater than 6 and about 7.5, between about 4.5 and about 5.7, between about 4.6 and about 5.7, between about 4.7 and about 5.7, between about 5.9 and about 7.5, between about 6.1 and about 7.5, between about 5.9 and about 6.4, between about 6.6 and about 7.5. In some cases, the pH of the foregoing compositions is not 4.5, not 4.6, not 5.8, not 6.0, or not 6.5.

In some cases, ascorbic acid in the foregoing compositions is present in a concentration that is about 20 mg/mL, about 30 mg/nL, about 40 mg/mL, about 50 mg/mL, about 60 ng/nL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some cases, ascorbic acid is present in a concentration between about 30 mg/mL and about 200 mg/mL, between about 40 mg/mL and about 200 mg/mL, between about 50 mg/mL and about 200 mg/mL, between about 75 mg/nL and about 200 mg/nL, between about 100 mg/mL and about 200 mg/mL, between about 110 mg/mL and about 200 mg/mL, between about 20 m/mL and about 49 mg/mL, or between about 21 mg/mL and about 49 mg/nL, between about 51 mg/nL and about 200 mg/mL, between about 51 mg/mL and about 199 mg/mL, between about 51 mg/mL and about 99 mg/mL, or between about 101 mg/mL and about 199 mg/mL. In some cases, the ascorbic acid concentration is not 20 mg/mL, not 50 mg/mL, not 100 mg/mL, not 200 mg/mL, or not 0.28 M.

In one embodiment, the pH of the foregoing compositions is not 5.8 and the concentration of ascorbic acid is not 0.28M. In another embodiment, the pH is not 5.8 and the concentration of ascorbic acid is not 50 mg/mL or not less than 50 mg/mL. In some cases, either of the foregoing compositions comprise water and ethanol. In some cases, the ethanol is present in less than about 5% by volume. In some cases, the ethanol is present in about 5% by volume, about 4% by volume, about 3% by volume, about 2% by volume, or about 1% by volume. In some cases, the ethanol is present in between about 0.1% and about 5% by volume.

### Imaging Agents and Related Methods

Imaging agents allow for the detection, imaging, and/or monitoring of the presence and/or progression of a condition, pathological disorder, and/or disease. Typically, an imaging agent is administered to a subject in order to provide information relating to at least a portion of the subject (e.g., human). In some cases, an imaging agent may be used to highlight a specific area of a subject, rendering organs, blood vessels, tissues, and/or other portions more detectable and more clearly imaged. By increasing the detectability and/or image quality of the object being studied, the presence and extent of disease and/or injury can be determined. An imaging agent may include a radioisotope for nuclear medicine imaging. The imaging agents of the invention typically comprise a radionuclide (or radioisotope).

The term “imaging agent” refers to a chemical compound that includes an imaging moiety. The compositions and methods as described herein comprise an imaging agent comprising pyridaben or pyridaben analog attached to an imaging moiety. The term “analog” is meant to include any compounds that are substantially similar in structure or atom connectivity to the referred structure or compound. These include compounds in which one or more individual atoms have been replaced, either with a different atom, or with a different functional group. The term analog implies a high degree of homology, but also may include compounds that are rationally derived from such a structure.

An “imaging moiety” refers to an atom or group of atoms that is capable of producing a detectable signal, optionally upon exposure to an external source of energy (e.g., electromagnetic radiation, ultrasound, and the like). Preferred imaging moieties are radionuclides (or radioisotopes). Non-limiting examples of imaging moieties include ¹¹C, ¹³N, ¹⁸F, ⁷⁶Br, ¹²³I, ¹²⁴I, ¹²⁵I, ¹³¹I, ^{99m}Tc, ⁹⁵Tc, ¹¹¹In, ⁶²Cu, ⁶⁴Cu, ⁶⁷Ga, and ⁶⁸Ga. In some embodiments, the imaging moiety is selected from the group consisting of ¹⁸F, ⁷⁶Br, ¹²⁴I, ¹³¹I, ⁶⁴Cu, ⁸⁹Zr, ^{99m}Tc, and ¹¹¹In. In certain embodiments, the imaging moiety is directly associated (i.e., through a covalent bond) with a compound as described herein (e.g., in the case of ¹⁸F, ⁷⁶Br, ¹²⁴I, or ¹³¹I). In other embodiments, the imaging moiety is associated with the compound through a chelator (e.g., in the case of ⁶⁴Cu, ⁸⁹Zr, ^{99m}Tc, and ¹¹¹In). Accordingly, for imaging moieties which are associated with a compound via a chelator, the term “imaging moiety” may also include the chelator. In certain embodiments, the imaging moiety is associated with the compound through non-covalent interactions (e.g., electrostatic interactions).

In some embodiments, a composition comprising imaging agents or a plurality of imaging agents is referred to as being enriched with an isotope such as a radioisotope. In such a case, the composition or the plurality may be referred to as being “isotopically enriched.” As an example, an “isotopically enriched” composition refers to a composition comprising a percentage of one or more isotopes of an element that is more than the naturally occurring percentage of that isotope. For example, a composition that is isotopically enriched with a fluoride species may be “isotopically enriched” with fluorine-18 (¹⁸F). Thus, with regard to a plurality of compounds, when a particular atomic position is designated as ¹⁸F, it is to be understood that the abundance (or frequency) of ¹⁸F at that position (in the plurality) is greater than the natural abundance (or frequency) of ¹⁸F, which is essentially zero.

In some embodiments, an atom designated as being enriched may have a minimum isotopic enrichment factor of about 0.001% (i.e., about 1 out of 10⁵ atoms is an enriched atom), 0.002%, 0.003%, 0.004%, 0.005%, 0.006%, 0.007%, 0.008%, 0.009%, 0.01%, about 0.05%, about 0.1%, about 0.2%, about 0.3%, about 0.4%, about 0.5%, about 0.75%, about 1%, about 2%, about 3%, about 4%, about 5%, about 10%, about 15%, about 20%, about 30%, about 40%, about 50%, about 60%, about 70%, about 80%, about 90%, about 95%, or greater. The minimum isotopic enrichment factor, in some instances, may range from about 0.001% to about 1%. For example, in embodiments wherein the imaging moiety is fluorine, a fluorine designated as ¹⁸F may have a minimum isotopic enrichment factor of about 0.001% (i.e., about 1 out of 10 fluorine species is ¹⁸F), 0.002, 0.003%, 0.004%, 0.005%, 0.006%, 0.007%, 0,008%, 0,009%, 0.01%, about 0.05%, about 0.1%, about 0.2%, about 0.3%, about 0.4%, about 0.5%, about 0.75%, about 1%, about 2%, about 3%, about 4%, about 5%, about 10%, about 15%, about 20%, about 30%, about 40%, about 50%, about 60%, about 70%, about 80%, about 90%, about 95%, or greater. Similarly, a plurality of imaging agents may be described as having a minimum isotopic enrichment factor of about 0.001% (i.e., about 1 out of 10⁵ imaging agents in the plurality comprises the desired isotope). Accordingly, similar enrichment factors as described above for compositions comprising imaging agents can be used to describe pluralities of imaging agents.

The isotopic enrichment of the compounds provided herein can be determined using conventional analytical methods known to one of ordinary skill in the art, including mass spectrometry and HPLC.

In some embodiments, an imaging agent comprising pyridaben or a pyridaben analog attached to an imaging moiety has a structure as in formula (I),

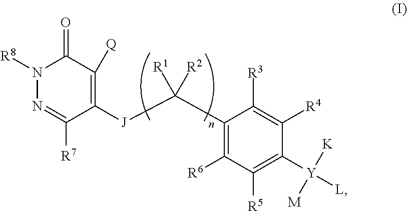

wherein:
J is selected from N(R⁹), S, O, C(═O), C(═O)O, NHCH₂CH₂O, a bond, or C(═O)N(R⁷);
when present, K is selected from hydrogen, alkoxyalkyl, alkyloxy, aryl, C₁-C₆ alkyl, heteroaryl, and an imaging moiety;
when present, L is selected from hydrogen, alkoxyalkyl, alkyloxy, aryl, C₁-C₆ alkyl, heteroaryl, and an imaging moiety;
M is selected from hydrogen, alkoxyalkyl, alkyloxy, aryl, C₁-C₆ alkyl, heteroaryl, and an imaging moiety; or
L and M, together with the atom to which they are attached, form a three-, four-, five-, or six-membered carbocyclic ring;
Q is halo or haloalkyl;
n is 0, 1, 2, or 3;
R¹, R², R⁷, and R⁸ are independently selected from hydrogen, C₁-C₆ alkyl, and an imaging moiety;
R³, R⁴, R⁵, and R⁶ are independently selected from hydrogen, halogen, hydroxyl, alkyloxy, C₁-C₆ alkyl, and an imaging moiety;
R⁸ is C₁-C₆ alkyl; and
Y is selected from a bond, carbon, and oxygen; provided that when Y is a bond, K and L are absent and M is selected from aryl and heteroaryl; and provided that when Y is oxygen, K and L are absent and M is selected from hydrogen, alkoxyalkyl, aryl, C₁-C₆ alkyl, and heteroaryl;
wherein each occurrence of alkoxyalkyl, alkyloxy, aryl, C₁-C₆ alkyl, and heteroaryl is optionally substituted with an imaging moiety,
provided that at least one imaging moiety is present in formula (I).

In some embodiments, R¹, R², R³, R⁴, R⁵, R⁶, R⁷, and R⁹ are independently selected from hydrogen, C₁-C₆ alkyl, and an imaging moiety; and R⁸ is C₁-C₆ alkyl.

In some embodiments, J is O; M is alkoxyalkyl, alkyloxy, aryl, C₁-C₆ alkyl, or heteroaryl, each optionally substituted with an imaging moiety; Q is halo or haloalkyl; n is 1; and R⁸ is C₁-C₆ alkyl.

In some embodiments, J is O; M is alkyloxy substituted with an imaging moiety; Q is halo; n is 1; and R⁸ is C₁-C₆ alkyl,

In some embodiments, J is O; and R⁸ is tert-butyl.

In some embodiments, J is O. In some embodiment, J is S.

In some embodiments, M is alkyloxy substituted with an imaging moiety.

In some embodiments, Y is carbon, K and L are hydrogen, and M is alkoxyalkyl, alkyloxy, aryl, C₁-C₆ alkyl, or heteroaryl, each optionally substituted with an imaging moiety. In some embodiments, Y is carbon, K and L are hydrogen, and M is alkyloxy substituted with an imaging moiety. In some embodiments, Y is carbon, K and L are hydrogen, and M is ethoxy substituted with an imaging moiety. In some embodiments, Y is carbon, K and L are hydrogen, and M is —OCHCH₂¹⁸F.

In some embodiments, Q is halo. In some embodiments, Q is fluoro. In some embodiments, Q is chloro. In some embodiments, Q is iodo. In some embodiments, Q is bromo. In some embodiments, Q is haloalkyl.

In some embodiments, R¹ and R² are each hydrogen. In some embodiments, one of R¹ and R² is hydrogen. In some embodiments, R¹ and R² are independently hydrogen or C₁-C₆ alkyl. In some embodiments, neither R¹ nor R² is an imaging moiety.

In some embodiments, R³, R⁴, R⁵, and R⁶ are each hydrogen. In some embodiments, three of R³, R⁴, R⁵, and R⁶ are hydrogen. In some embodiments, two of R³, R⁴, R⁵, and R⁶ is hydrogen. In some embodiments, one of R³, R⁴, R⁵, and R⁶ are hydrogen. In some embodiments, each of R³, R⁴, R⁵, and R⁶ is independently hydrogen or C₁-C₆ alkyl. In some embodiments, none of R³, R⁴, R⁵, and R⁶ is an imaging moiety.

In some embodiments, R⁷ is hydrogen. In some embodiments R⁷ is C₁-C₆ alkyl. In some embodiments, R⁷ is not an imaging moiety.

In some cases R⁸ is methyl, ethyl, n-propyl, i-propyl, n-butyl, i-butyl, or tert-butyl, each may be optionally substituted with a leaving group. In some embodiments R⁸ is tert-butyl. In some embodiments, R⁸ is not tert-butyl.

In some embodiments, n is 0. In some embodiments, n is 1. In some embodiments, n is 2. In some embodiments, n is 3.

In some embodiments, the imaging moiety is a radioisotope such as may be used in nuclear medicine imaging, a paramagnetic species such as may be used in MR imaging, an echogenic entity such an as may be used in ultrasound imaging, a fluorescent entity such as may be used in fluorescence imaging, or a light-active entity such as may be used in optical imaging. In some embodiments, a paramagnetic species for use in MR imaging is Gd³⁺, Fe³⁺, In³⁺, or Mn²⁺. In some embodiments, an echogenic entity for use in ultrasound imaging is a surfactant encapsulated fluorocarbon microsphere. In some embodiments, a radioisotope for nuclear medicine imaging is ¹¹C, ¹³N, ¹⁸F, ¹²³I, ¹²⁵I, ^{99m}Tc, ⁹⁵Tc, ¹¹¹In, ⁶²Cu, ⁶⁴Cu, ⁶⁷Ga, or ⁶⁸Ga.

In some embodiments, the imaging moiety is ¹⁸F.

In some embodiments, the imaging agent is selected from the group consisting of

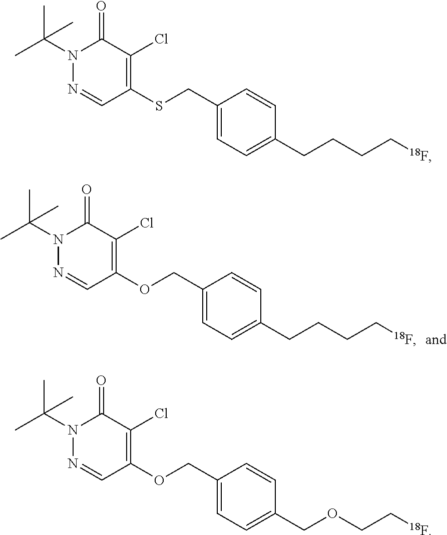

In some embodiments, the imaging agent is:

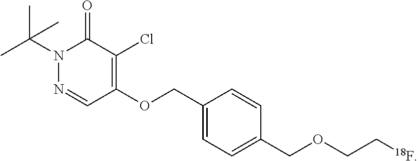

In some embodiments, the imaging agent may be pharmaceutically acceptable. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The imaging agents may also be present as pharmaceutically acceptable salts. The pharmaceutically acceptable salt may be a derivative of a disclosed compound wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

In some cases, an imaging agent of formula (I) may be synthesized using an automated synthesis module. Automated synthesis modules will be known to those of ordinary skill in the art. In some cases, an imaging agent may be synthesized according to the teachings of automated synthesis modules described in International Patent Publication No. WO2011/097649, published Aug. 11, 2011, the teachings of which relating to automated synthesis modules being incorporated by reference herein.

In some embodiments, the diagnostic compositions described herein may find application in methods of imaging, including methods of imaging a subject that includes administering a diagnostic composition as described herein, and imaging a region of the subject that is of interest. Regions of interest may include, but are not limited to, the heart, cardiovascular system, cardiac vessels, blood vessels (e.g., arteries, veins) brain, and other organs. A parameter of interest, such as blood flow, cardiac wall motion, etc. can be imaged and detected using methods and/or systems of the invention. In some aspects of the invention, methods for evaluating perfusion, including myocardial perfusion, are provided. In all embodiments, the subject includes a human subject.

In some embodiments, a method of imaging includes (a) administering to a subject a diagnostic composition that includes an imaging agent, and (b) acquiring at least one image of at least a portion of the subject. In some cases, acquiring employs positron emission tomography (PET) for visualizing the distribution of the imaging agent within at least a portion of the subject. As will be understood by those of ordinary skill in the art, imaging may include full body imaging of a subject, or imaging of a specific body region or tissue of the subject that is of interest. For example, if a subject is known to have, or is suspected of having myocardial ischemia, methods may be used to image the heart of the subject. In some embodiments, imaging may be limited to the heart, or may include the heart and its associated vascular system.

In some embodiments, a method may include diagnosing or assisting in diagnosing a disease or condition, assessing efficacy of treatment of a disease or condition, or imaging in a subject with a known or suspected disease or condition. A disease can be any disease of the heart or other organ or tissue nourished by the vascular system. In some embodiments, the disease or condition is a cardiovascular disease or condition. The vascular system includes coronary arteries, and all peripheral arteries supplying nourishment to the peripheral vascular system and the brain, as well as veins, arterioles, venules, and capillaries. Examples of cardiovascular diseases include diseases of the heart, such as coronary artery disease, myocardial infarction, myocardial ischemia, angina pectoris, congestive heart failure, cardiomyopathy (congenital or acquired), arrhythmia, or valvular heart disease. In some embodiments, the methods disclosed herein are useful for monitoring and measuring coronary artery disease and/or myocardial perfusion. For example, a method may determine the presence or absence of coronary artery disease and/or the presence or absence of myocardial infarct. Conditions of the heart may include damage, not brought on by disease but resulting from injury—e.g., traumatic injury, surgical injury. In some cases, methods may include determining a parameter of, or the presence or absence of, myocardial ischemia, rest (R) and/or stress (S) myocardial blood flows (MBFs), coronary flow reserve (CFR), coronary artery disease (CAD), left ventricular ejection fraction (LVEF), end-systolic volume (ESV), end-diastolic volume (EDV), and the like.

### Definitions

For convenience, certain terms employed in the specification, examples, and appended claims are listed here.

Definitions of specific functional groups and chemical terms are described in more detail below. For purposes of this invention, the chemical elements are identified in accordance with the Periodic Table of the Elements. CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry. Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by assymetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

As used herein, the term “alkyl” is given its ordinary meaning in the art and refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some cases, the alkyl group may be a lower alkyl group, i.e., an alkyl group having 1 to 10 carbon atoms (e.g., methyl, ethyl, propyl, butyl, pentyl, hexyl, heptyl, octyl, nonyl, or decyl). In some embodiments, a straight chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, t-butyl, cyclobutyl, hexyl, and cyclohexyl.

The term “alkylene” as used herein refers to a bivalent alkyl group. An “alkylene” group is a polymethylene group, i.e., —(CH₂)_{z}—, wherein z is a positive integer, e.g., from 1 to 20, from 1 to 10, from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described herein for a substituted aliphatic group.

The terms “alkenyl” and “alkynyl” are given their ordinary meaning in the art and refer to unsaturated aliphatic groups analogous in length and possible substitution to the alkyls described above, but that contain at least one double or triple bond respectively

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, t-butyl, n-pentyl, sec-pentyl, isopentyl, t-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “cycloalkyl,” as used herein, refers specifically to groups having three to ten, preferably three to seven carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl”, “heteroalkynyl”, and the like. Furthermore, as used herein, the terms “heteroalkyl”, “heteroalkenyl”, “heteroalkynyl”, and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroalkyl” is given its ordinary meaning in the art and refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, poly(ethylene glycol), and alkyl-substituted amino.

The terms “heteroalkenyl” and “heteroalkynyl” are given their ordinary meaning in the art and refer to unsaturated aliphatic groups analogous in length and possible substitution to the heteroalkyls described above, but that contain at least one double or triple bond respectively.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CHF₂; —CH₂F; —CH₂CF₃; —CHCl₂; —CH₂OH; —CH₂CH₂OH: —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alycyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

The term “aryl” is given its ordinary meaning in the art and refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. Substituents include, but are not limited to, any of the previously mentioned substituents, i.e., the substituents recited for aliphatic moieties, or for other moieties as disclosed herein, resulting in the formation of a stable compound. In some cases, an aryl group is a stable mono- or polycyclic unsaturated moiety having preferably 3-14 carbon atoms, each of which may be substituted or unsubstituted. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl groups.

The terms “heteroaryl” is given its ordinary meaning in the art and refers to aryl groups comprising at least one heteroatom as a ring atom. A “heteroaryl” is a stable heterocyclic or polyheterocyclic unsaturated moiety having preferably 3-14 carbon atoms, each of which may be substituted or unsubstituted. Substituents include, but are not limited to, any of the previously mentioned substituents, i.e., the substituents recited for aliphatic moieties, or for other moieties as disclosed herein, resulting in the formation of a stable compound. In some cases, a heteroaryl is a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will also be appreciated that aryl and heteroaryl moieties, as defined herein may be attached via an alkyl or heteroalkyl moiety and thus also include -(alkyl)aryl, -(heteroalkylaryl, -(heteroalkyl)heteroaryl, and -(heteroalkyl)heteroaryl moieties. Thus, as used herein, the phrases “aryl or heteroaryl moieties” and “aryl, heteroaryl, -(alkyl)aryl, -(heteroalkyl)aryl, -(heteroalkyl)heteroaryl, and -(heteroalkyl)heteroaryl” are interchangeable. Substituents include, but are not limited to, any of the previously mentioned substituents, i.e., the substituents recited for aliphatic moieties, or for other moieties as disclosed herein, resulting in the formation of a stable compound.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; OH; —NO₂; —CN; —CF₃; —CH₂F; —CHF₂; —CH₂CF₃; —CHCl₂; —CH₂OH; —CH₂CH₂OH; —CH₂NH; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments described herein.

The term “heterocycle” is given its ordinary meaning in the art and refers to refer to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms.

The term “heterocycle” may include heteroaryl groups, saturated heterocycles (e.g., cycloheteroalkyl) groups, or combinations thereof. The heterocycle may be a saturated molecule, or may comprise one or more double bonds. In some cases, the heterocycle is a nitrogen heterocycle, wherein at least one ring comprises at least one nitrogen ring atom. The heterocycles may be fused to other rings to form a polycyclic heterocycle. The heterocycle may also be fused to a spirocyclic group. In some cases, the heterocycle may be attached to a compound via a nitrogen or a carbon atom in the ring.

Heterocycles include, for example, thiophene, benzothiophene, thianthrene, furan, tetrahydrofuran, pyran, isobenzofuran, chromene, xanthene, phenoxathiin, pyrrole, dihydropyrrole, pyrrolidine, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, triazole, tetrazole, oxazole, isoxazole, thiazole, isothiazole, phenanthridine, acridine, pyrimidine, phenanthroline, phenazine, phenarsazine, phenothiazine, furazan, phenoxazine, pyrrolidine, oxolane, thiolane, oxazole, oxazine, piperidine, homopiperidine (hexamethyleneimine), piperazine (e.g., N-methyl piperazine), morpholine, lactones, lactams such as azetidinones and pyrrolidinones, sultams, sultones, other saturated and/or unsaturated derivatives thereof, and the like. The heterocyclic ring can be optionally substituted at one or more positions with such substituents as described herein. In some cases, the heterocycle may be bonded to a compound via a heteroatom ring atom (e.g., nitrogen). In some cases, the heterocycle may be bonded to a compound via a carbon ring atom. In some cases, the heterocycle is pyridine, imidazole, pyrazine, pyrimidine, pyridazine, acridine, acridin-9-amine, bipyridine, naphthyridine, quinoline, benzoquinoline, benzoisoquinoline, phenanthridine-1,9-diamine, or the like.

The terms “halo” and “halogen” as used herein refer to an atom selected from the group consisting of fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino,” as used herein, refers to a primary (—NH₂), secondary (—NHRₓ), tertiary (—NRₓR_{y}), or quaternary (—N⁺RₓR_{y}R_{z}) amine, where Rₓ, R_{y}, and R_{z} are independently an aliphatic, alicyclic, heteroaliphatic, heterocyclic, aryl, or heteroaryl moiety, as defined herein. Examples of amino groups include, but are not limited to, methylamino, dimethylamino, ethylamino, diethylamino, methylethylamino, iso-propylamino, piperidino, trimethylamino, and propylamino.

The term “alkyne” is given its ordinary meaning in the art and refers to branched or unbranched unsaturated hydrocarbon groups containing at least one triple bond. Non-limiting examples of alkynes include acetylene, propyne, 1-butyne, 2-butyne, and the like. The alkyne group may be substituted and/or have one or more hydrogen atoms replaced with a functional group, such as a hydroxyl, halogen, alkoxy, and/or aryl group.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, t-butoxy, neopentoxy and n-hexoxy. Examples of thioalkyl include, but are not limited to, methylthio, ethylthio, propylthio, isopropylthio, n-butylthio, and the like.

The term “aryloxy” refers to the group, —O-aryl.

The term “acyloxy” refers to the group, —O-acyl.

The term “alkoxyalkyl” refers to an alkyl group substituted with at least one alkoxy group (e.g., one, two, three, or more, alkoxy groups). For example, an alkoxyalkyl group may be —(C₁₋₆-alkyl)-O—(C₁₋₆-alkyl), optionally substituted. In some cases, the alkoxyalkyl group may be optionally substituted with another alkyoxyalkyl group (e.g., —(C₁₋₆-alkyl)-O—(C₁₋₆-alkyl)-O—(C₁₋₆-alkyl), optionally substituted.

It will be appreciated that the above groups and/or compounds, as described herein, may be optionally substituted with any number of substituents or functional moieties. That is, any of the above groups may be optionally substituted. As used herein, the term “substituted” is contemplated to include all permissible substituents of organic compounds, “permissible” being in the context of the chemical rules of valence known to those of ordinary skill in the art. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful for the formation of an imaging agent or an imaging agent precursor. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkyithio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like. In some embodiments, as noted herein, the substituent may be an imaging moiety, for example, ⁵⁸F.

As used herein, the term “determining” generally refers to the analysis of a species or signal, for example, quantitatively or qualitatively, and/or the detection of the presence or absence of the species or signals.

The term “diagnostic imaging,” as used herein, refers to a procedure used to detect an imaging agent.

The term “diagnosis” as used herein encompasses identification, confirmation, and/or characterization of a condition, a disease, and/or a disorder.

As used herein, the term “subject” refers to a human or non-human mammal or animal. Non-human mammals include livestock animals, companion animals, laboratory animals, and non-human primates. Non-human subjects also specifically include, without limitation, horses, cows, pigs, goats, dogs, cats, mice, rats, guinea pigs, gerbils, hamsters, mink, and rabbits. In some embodiments of the invention, a subject is referred to as a “patient.” In some embodiments, a patient or subject may be under the care of a physician or other health care worker, including, but not limited to, someone who has consulted with, received advice from or received a prescription or other recommendation from a physician or other health care worker.

As used herein, a “portion of a subject” refers to a particular region of a subject, location of the subject. For example, a portion of a subject may be the brain, heart, vasculature, cardiac vessels, tumor, etc., of a subject.

Any of the compounds described herein may be in a variety of forms, such as, but not limited to, salts, solvates, hydrates, tautomers, and isomers.

In certain embodiments, the imaging agent is a pharmaceutically acceptable salt of the imaging agent. The term “pharmaceutically acceptable salt” as used herein refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et ad, describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, loweralkyl sulfonate and aryl sulfonate.

In certain embodiments, the compound is in the form of a hydrate or solvate. The term “hydrate” as used herein refers to a compound non-covalently associated with one or more molecules of water. Likewise, the term “solvate” refers to a compound non-covalently associated with one or more molecules of an organic solvent.

In certain embodiments, the compound described herein may exist in various tautomeric forms. The term “tautomer” as used herein includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol; amide-to-imide; lactam-to-lactim; enamine-to-imine; and enamine-to-(a different) enamine tautomerizations.

In certain embodiments, the compounds described herein may exist in various isomeric forms. The term “isomer” as used herein includes any and all geometric isomers and stereoisomers (e.g., enantiomers, diasteromers, etc.). For example, “isomer” includes cis- and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically-enriched,” as used herein, means that the compound is made up of a significantly greater proportion of one enantiomer. In certain embodiments the compound of the present invention is made up of at least about 90% by weight of a preferred enantiomer. In other embodiments the compound is made up of at least about 95%, 98%, or 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, IN 1972).

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Preparation of [¹⁸F]fluoride

[¹⁸F]Fluoride was produced by proton bombardment of [¹⁸O]H₂O in a cyclotron; the nuclear chemical transformation is shown below and may be summarized as ¹⁸O(p,n)¹⁸F. For purposes of the bombardment, the chemical form of the ¹⁸O is H₂¹⁸O. The chemical form of the resulting ¹⁸F is fluoride ion.

^{˜}O+proton→¹⁸F+neutron

According to established industry procedures, [¹⁸O]H₂O (2-3 mL) housed within the cyclotron target, was bombarded with 11 MeV protons (nominal energy); where the proton threshold energy for the reaction is 2.57 MeV and the energy of maximum cross section is 5 MeV. Target volume, bombardment time and proton energy each may be adjusted to manage the quantity of [¹⁸F]fluoride produced.

### Example 2

### Preparation of an Imaging Agent Precursor—Acetontrile Concentrate

An imaging agent precursor having the structure:

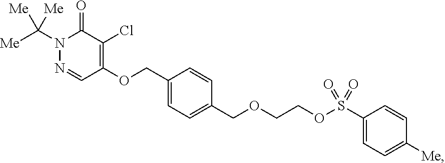 [SMILES: Cc1ccc(S(=O)(=O)OCCOCc2ccc(COc3cnn(C(C)(C)C)c(=O)c3Cl)cc2)cc1]

(20.4 g, 39.2 mmol), was dissolved in anhydrous MeCN (3400 mL) then transferred through an Opticap XL2 Durapore filter (0.2 μm) into 5 mL glass vials; 2.0 mL fill volume. The vials were then fitted with rubber septa, sealed with an aluminum crimp and stored at ambient temperature prior to use.

### Example 3

### General Preparation of an Imaging Agent

An imaging agent having the structure:

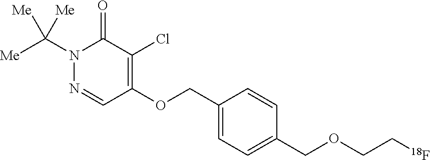 [SMILES: CC(C)(C)n1ncc(OCc2ccc(COCC[18F])cc2)c(Cl)c1=O]

was prepared using the general method of nucleophilic substitution between [¹⁸F]fluoride and the imaging agent precursor of Example 2 as known by those skilled in the art. Specific details of the various experimental methods are provided in the examples which follow.

### Example 4

### Preparation of an Imaging Agent

Aqueous [¹⁸F]fluoride, as prepared in Example 1, was filtered through an anion exchange column to remove unreacted [¹⁸O]H₂O; [¹⁸F]fluoride was retained within the cationic resin matrix. Potassium carbonate (K₂CO₃, 10 ng) was then dissolved in H₂O (1 mL) and mixed with a solution of Kryptofix® 222 (4,7,13,16,21,24-hexaoxa-1,10-diazabicyclo[8.8.8]-hexacosane) in anhydrous acetonitrile (CH₃CN, 4 mL); an aliquot of the resulting solution (1 mL) was used to elute [¹⁸F]fluoride from the resin. The radioactivity content of the eluent was determined and the resulting solution transferred to the reaction vessel of the Explora RN Chemistry Module with control applied using the GINA-Star software package. The eluent was then concentrated to dryness (70-95° C., argon bleed; partial vacuum (250-12 mbar)) then treated with the acetonitrile solution of the imaging agent precursor as prepared in Example 2. The resulting mixture was heated to 90° C. and maintained 10 min.

After cooling, the acetonitrile was evaporated (55° C., argon bleed; partial vacuum (250-15 mbar)) and the resulting mixture suspended in mobile phase (40% 50 mM aqueous NH₄OAc/60% MeCN, 1.3 mL). The solution was then loaded into a sample loop and purified by HPLC using a Phenomenex Synergi 4 μHydro-RP C18, (10×250 mm) using a 40:60 50 mM NH₄OAc/MeCN eluent at a flow rate of 5 mL/min. The imaging agent having the structure:

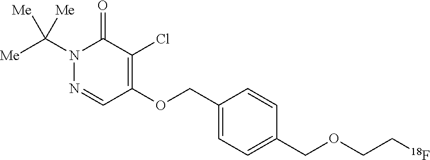 [SMILES: CC(C)(C)n1ncc(OCc2ccc(COCC[18F])cc2)c(Cl)c1=O]

was then collected, diluted with an ascorbic acid solution (10-15 mL), then passed through a C18 Sep-Pak® cartridge, previously conditioned with 10 mL of ethanol followed by 10 mL of an ascorbic acid solution; The imaging agent was retained within the C18 resin matrix and the filtrate discarded. The cartridge was then successively washed with an ascorbic acid solution (10 mL), the filtrate discarded, then absolute ethanol (≤0.5 mL) and the filtrate collected. The resulting ethanol concentrate of the imaging agent was then diluted with an ascorbic acid solution prior to use.

### Example 5

### Stability of Radiopharmaceutical Compositions

The radiochemical purity (RCP) of a labeled compound (i.e., as in Example 3) is known to be dependent on certain conditions of its preparation including, but not limited to, reaction temperature, solution pH and overall synthesis time. Once prepared with high RCP, the labeled compound is formulated into a radiopharmaceutical composition designed to stabilize the labeled compound over time. Certain radiopharmaceutical compositions of the present invention are effective in maintaining the stability of labeled compounds for up to 12 h.

Both chemical integrity and overall stability of a radiopharmaceutical composition is measured through determination of the change in RCP of the labeled compound over time using ITLC or more preferably HPLC. The advantage of using HPLC is that impurities caused by radiolytic degradation may be readily separated from the labeled compound under certain chromatographic conditions. Improved stability profiles for radiopharmaceutical compositions may thus be demonstrated by observing changes in the HPLC profile of the composition over time. Several HPLC methods have been developed for monitoring the stability of radiopharmaceutical compositions of the present invention:

HPLC Method A: Analytical HPLC was performed on an Agilent Technologies 1100 LC containing a radiometric detection system. Radiochemical impurities were evaluated using a Berthold radiation detector and a Waters Zorbax SB-C18 column (4.6×50 mm, 1.8 μm) using an isocratic elution (45:55 H₂O/MeCN) at 1 mL/min.

HPLC Method B: Analytical HPLC was performed on an Agilent Technologies 1100 LC containing a spectrophotometric detection system. Non-radiochemical impurities were evaluated at 295 nm using a Waters Zorbax SB-C18 column (4.6×50 mm, 1.8 μm) with an 8%/min gradient from 20-100% MeCN containing 0.1% formic acid and 10% H₂O at 1 mL/min.

HPLC Method C: Analytical HPLC was performed on an Agilent Technologies 1100 LC containing both radiometric and spectrophotometric detection systems. Radiochemical impurities were evaluated using a Raytest GabiStar radiation detector and non-radiochemical impurities were evaluated at 295 nm both using a Waters Zorbax SB-C18 column (4.6×50 mm, 1.8 μm) with a 6%/min gradient from 20-50% MeCN, followed by a 1.4%/min gradient from 50-60% MeCN, followed by a 2%/min gradient from 60-70% MeCN each containing 0.1% formic acid and 10% H₂O at 1 mL/min.

HPLC Method D: Analytical HPLC was performed on an Agilent Technologies 1100 LC containing both radiometric and spectrophotometric detection systems. Radiochemical impurities were evaluated using a Raytest GabiStar radiation detector and non-radiochemical impurities were evaluated at 295 nm both using a Waters Zorbax SB-C18 column (4.6×50 mm, 1.8 μm) with a 30%/min gradient from 30-60% MeCN, followed by a 2 min isocratic hold at 60% MeCN, followed by a 5%/mm gradient from 60-80% MeCN each containing 0.1% trifluoroacetic acid and 10% 1-120 at 1 mL/min.

### Example 6

### pH and the Stability of an Imaging Agent

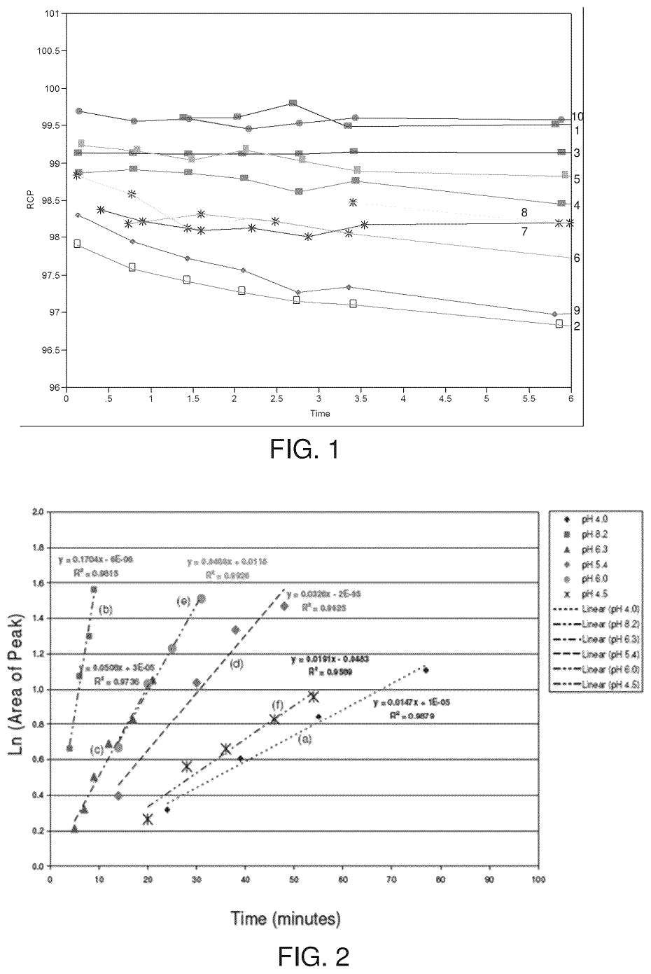

The stability of radiopharmaceutical compositions containing an imaging agent was assessed over a range of pH values. A series of ascorbic acid solutions were prepared with unique pH values (Table 1) by the addition of either aqueous hydrochloric acid or sodium hydroxide to a stock solution of sodium ascorbate in H₂O. Each solution was then utilized in the preparation of the imaging agent as described in Example 4, and the resulting compositions monitored for changes in radiochemical purity over time using the HPLC methods described in Example 5. Results for the 10 solutions are plotted in FIG. 1.

TABLE 1
pH values of ascorbic acid solutions

| Entry | pH Value |
|---|---|
| 1 | 4.0 |
| 2 | 5.8 |
| 3 | 4.0 |
| 4 | 4.0 |
| 5 | 4.5 |
| 6 | 4.6 |
| 7 | 4.6 |
| 8 | 4.6 |
| 9 | 6.5 |
| 10 | 2.4 |

As the data from FIG. 1 indicate, both the initial RCP of the resulting radiopharmaceutical compositions and the change in RCP over time was directly dependent upon the initial pH of the ascorbic acid solution. Solutions with higher pH values (closer to physiological pH of 7-7.5) had markedly less initial stability and stability to storage than did those with relatively more acidic compositions. In particular, the two lowest plots on the graph were derived from compositions prepared at pH 5.8 and 6.5 respectively.

### Example 7

### pH and the Chemical Integrity of an Imaging Agent

The chemical integrity of radiopharmaceutical compositions containing the non-radioactive congener of the imaging agent (2-(tert-butyl)-4-chloro-5-((4-((2-fluoroethoxy)methyl)benzyl)oxy)pyridazin-3(2H)-one) was assessed over a range of pH values. A series of ascorbic acid solutions (50 mg/mL) were prepared with unique pH values by the addition of either aqueous hydrochloric acid or sodium hydroxide to a stock solution of sodium ascorbate in H₂O (FIG. 2). Each solution was then utilized in the preparation of radiopharmaceutical compositions containing the non-radioactive congener of the imaging agent (5 ag/mL) and ethanol (5%). The resulting solutions were treated with [¹⁸F]NaF then monitored for changes in chemical purity (non-radioactive impurities) over time using the HPLC methods outlined in Example 5. As the data in FIG. 2 indicate, a first order reaction rate was observed for the formation certain non-radioactive impurities in the composition. A ten-fold reduction in the rate of impurity formation occurred over the range of pH values considered.

### Example 8

### Concentration of Ascorbic Acid and the Stability of an Imaging Agent

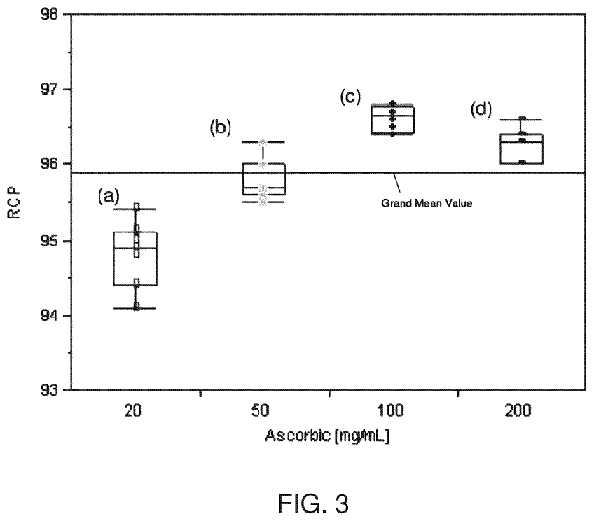

The stability of radiopharmaceutical compositions containing an imaging agent was assessed over a range of ascorbic acid concentration values. A series of ascorbic acid solutions were prepared with unique concentration values (20-200 mg/mL; pH 5.8) through serial dilution from a stock concentration of 500 mg/mL. Each solution was then utilized in the preparation of the imaging agent described in Example 4, and the resulting compositions monitored for changes in radiochemical purity over time using the HPLC methods described in Example 5. As the data in FIG. 3 indicate, both the initial RCP and the variability in RCP over time do not significantly change over the 200 to 50 mg/mL range; an overall decrease in RCP was however observed at the 20 mg/mL level.

### Example 9

### Preparation of an Imaging Agent

Aqueous [¹⁸F]fluoride, as prepared in Example 1, was filtered through an anion exchange column to remove unreacted [¹⁸O]H₂O; [¹⁸F]fluoride was retained within the cationic resin matrix. The column was then washed with aqueous Et₄NHCO₃ with transfer to the reaction vessel. The resulting solution was diluted with MeCN then concentrated to dryness using elevated temperature and reduced pressure. The mixture of anhydrous [¹⁸F]Et₄NF and Et₄NHCO₃ thus obtained was treated with the acetonitrile solution of the imaging agent precursor as prepared in Example 2, then warmed to 85-120° C. and maintained 5-20 min. After cooling, the solution was diluted with H₂O or H₂O/MeCN then directly purified by HPLC using a 45:55 H₂O/MeCN eluent. The main product peak was collected and diluted with ascorbic acid (10 mL of a 0.28 M solution; pH 2).

The resulting solution was filtered through a C18 Sep-Pak® cartridge to remove MeCN; The imaging agent having the structure:

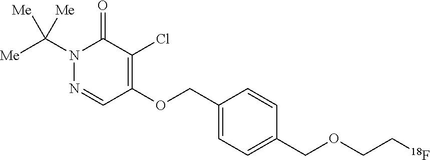 [SMILES: CC(C)(C)n1ncc(OCc2ccc(COCC[18F])cc2)c(Cl)c1=O]

was retained within the C18 resin matrix and the filtrate discarded. The cartridge was successively washed with ascorbic acid (10 mL, of a 0.28 M solution in H₂O; pH 2), the filtrate discarded, then absolute EtOH (≤0.50 mL), and the filtrate collected. The ethanol concentrate of the imaging agent thus obtained was further diluted with ascorbic acid (10.0 nL; pH 5.8) then filtered through a Millipore Millex GV PVDF sterilizing filter (0.22 μm×13 mm).

### Example 10

### pH, Radioactivity Concentration and the Stability of an Imaging Agent

The stability of radiopharmaceutical compositions containing an imaging agent was assessed over a range of ascorbic acid and radioactivity concentration values. A series of ascorbic acid solutions were prepared with unique concentration values (30-50 mg/mL; pH 5.8) through serial dilution from a stock concentration of 500 ng/mL. Each solution was then utilized in the preparation of the imaging agent as described in Example 9, and the resulting compositions monitored for changes in radiochemical purity over time using the HPLC methods described in Example 5. As the data in Table 2 indicate, both the initial RCP and the variability in RCP over time do not significantly change over the 30 to 50 mg/nL and 30 to 115 mCi/mL range tested. Each radiopharmaceutical composition maintained an RCP value ≥95% for the duration of the study.

TABLE 2
Stability of radiopharmaceutical compositions

| | Ascorbic Acid | Radioactive | | | | | |
|---|---|---|---|---|---|---|---|
| Synthesis | Concentration | Concentration | Radiochemical Purity | | | | |
| Module | (mg/mL) | (mCi/mL) | 0 h | 3 h | 6 h | 9 h | 12 h |
| Siemens GN | 30 | 29.5 | 100.0 | 100.0 | 100.0 | 100.0 | 100.0 |
| Eckert & Ziegler | 30 | 68.0 | 100.0 | 97.6 | 96.7 | 96.2 | 96.1 |
| Eckert & Ziegler | 50 | 44.8 | 100.0 | 100.0 | 100.0 | 100.0 | 100.0 |
| Siemens GN | 50 | 47.0 | 100.0 | 100.0 | 99.2 | 99.4 | 99.5 |
| Siemens RN | 50 | 50.0 | 100.0 | 98.2 | 99.0 | 98.0 | 98.2 |
| GE MX | 50 | 65.4 | 100.0 | 100.0 | 98.7 | 99.3 | 96.9 |
| Siemens GN | 50 | 115.9 | 99.7 | 97.1 | 97.1 | 96.0 | 96.0 |

### Example 11

### Preparation of an Imaging Agent using the Explora RN Synthesis Module

The product of Example 1 was filtered through an anion exchange column to remove unreacted [¹⁸O]H₂O; [¹⁸F]fluoride was retained within the cationic resin matrix. The column was then washed with Et₄NHCO₃ (5.75 μmol; 0.500 mL of a 11.5 mM solution in H₂O) with transfer to the reaction vessel. The resulting solution was diluted with MeCN (0.500 mL) then concentrated to dryness; 150 mm Hg at 115° C. for 4 min. The mixture of anhydrous [¹⁸F]Et₄NF and Et₄NHCO₃ thus obtained was treated with the imaging agent precursor of Example 2 (11.5 μmol; 1.00 mL of a 11.5 mM solution in MeCN) then warmed to 90° C. and maintained 20 min. After cooling to 35° C., the solution was diluted with H₂O (1.00 mL) then directly purified by HPLC on a Waters Xterra MS C18 column (10 μm; 10×250 mm) using a 45:55 H₂O/MeCN eluent at a flow rate of 5 mL/min. The main product peak eluting at 11 min was collected and diluted with ascorbic acid (10 mL of a 0.28 M solution in H₂O; pH 2).

The resulting solution was filtered through a C18 Sep-Pak® cartridge to remove MeCN; the imaging agent having the structure:

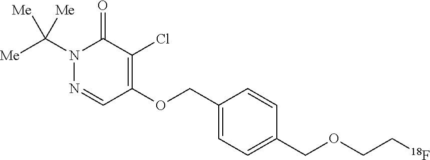 [SMILES: CC(C)(C)n1ncc(OCc2ccc(COCC[18F])cc2)c(Cl)c1=O]

was retained within the C18 resin matrix and the filtrate discarded. The cartridge was successively washed with ascorbic acid (10 mL of a 0.28 M solution in H₂O; pH 2), the filtrate discarded, then absolute EtOH (0.50 mL), and the filtrate collected. The ethanol concentrate of the imaging agent thus obtained was further diluted with ascorbic acid (10.0 mL of a 0.28 M solution in H₂O; pH 5.8) then filtered through a Millipore Millex GV PVDF sterilizing filter (0.22 μm×13 mm); 58% decay corrected radiochemical yield.

In another case, similar steps and conditions were employed as above except the Et₄NHCO₃ was 11.5 μmol (0.500 mL of a 23.0 mM solution in H₂O); the solution was concentrated to dryness at 280 mbar, 95-115° C., 4 min; the mixture of anhydrous [¹⁸F]Et₄NF and Et₄NHCO₃ treated with the imaging agent precursor of Example 2 was warmed to 90° C. and maintained 10 min; and the product had 61% decay corrected radiochemical yield.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying.” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A composition, comprising:
an imaging agent comprising pyridaben or a pyridaben analog attached to an imaging moiety; and
ascorbic acid,
wherein the pH of the composition is between about 1.5 and 3.5 and wherein ascorbic acid is present at a concentration between about 20 mg/mL and about 200 mg/mL ascorbic acid.

2-8. (canceled)

9. The composition of claim 1, wherein the composition further comprises water.

10. The composition of claim 1, wherein the composition further comprises acetonitrile.

11. A diagnostic composition, comprising:
an imaging agent comprising pyridaben or a pyridaben analog attached to an imaging moiety; and
ascorbic acid,
wherein the pH of the composition is between about 4.5 and 7.5, and wherein ascorbic acid is present in a concentration between about 20 mg/mL and about 200 mg/mL.

12-18. (canceled)

19. The composition of claim 11, further comprising water.

20. The composition of claim 11, further comprising an alcohol.

21. The composition of claim 20, wherein the alcohol is ethanol.

22. The composition of claim 21, wherein ethanol is present in less than about 5% by volume.

23. The composition of claim 21, wherein ethanol is present in about 5% by volume, or about 4% by volume, or about 3% by volume, or about 2% by volume, or about 1% by volume.

24. The composition of claim 11, wherein the radioactive concentration of the composition is about 1 mCi/mL, about 2 mCi/mL, about 3 mCi/mL, about 4 mCi/mL, about 5 mCi/mL, about 6 mCi/mL, about 7 mCi/mL, about 8 mCi/mL, about 9 mCi/mL, or about 10 mCi/mL.

25. The composition claim 1, wherein the radioactive concentration of the composition is between about 1 mCi/mL and about 200 mCi/mL.

26. The composition claim 11, wherein the radioactive concentration of the composition is less than or equal to about 65 mCi/mL.

27. The composition of claim 1, wherein the composition has a radiochemical purity of at least about 95%.

28. (canceled)

29. The composition of claim 27, wherein the composition has a radiochemical purity of at least 95% for at least 12 hours.

30. (canceled)

31. The composition of claim 1, wherein the imaging agent has a structure as in formula (I), 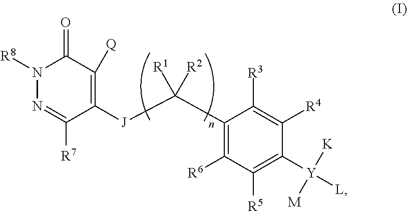
wherein:
J is selected from N(R⁹), S, O, C(═O), C(═O)O, NHCH₂CH₂O, a bond, or C(═O)N(R⁷);
when present, K is selected from hydrogen, alkoxyalkyl, alkyloxy, aryl, C₁-C₆ alkyl, heteroaryl, and an imaging moiety;
when present, L is selected from hydrogen, alkoxyalkyl, alkyloxy, aryl, C₁-C₆ alkyl, heteroaryl, and an imaging moiety;
M is selected from hydrogen, alkoxyalkyl, alkyloxy, aryl, C₁-C₆ alkyl, heteroaryl, and an imaging moiety; or
L and M, together with the atom to which they are attached, form a three-, four-, five-, or six-membered carbocyclic ring;
Q is halo or haloalkyl;
n is 0, 1, 2, or 3;
R¹, R², R⁷, and R⁹ are independently selected from hydrogen, C₁-C₆ alkyl, and an imaging moiety;
R³, R⁴, R⁵, and R⁶ are independently selected from hydrogen, halogen, hydroxyl, alkyloxy, C₁-C₆ alkyl, and an imaging moiety;
R⁸ is C₁-C₆ alkyl; and
Y is selected from a bond, carbon, and oxygen; provided that when Y is a bond, K and L are absent and M is selected from aryl and heteroaryl; and provided that when Y is oxygen, K and L are absent and M is selected from hydrogen, alkoxyalkyl, aryl, C₁-C₆ alkyl, and heteroaryl;
wherein each occurrence of alkoxyalkyl, alkyloxy, aryl, C₁-C₆ alkyl, and heteroaryl is optionally substituted with an imaging moiety,
provided that at least one imaging moiety is present in formula (I).

32. The composition of claim 31, wherein:
J is O;
M is selected from alkoxyalkyl, alkyloxy, aryl, C₁-C₆ alkyl, and heteroaryl, each optionally substituted with an imaging moiety;
Q is halo or haloalkyl;
n is 1; and
R⁸ is C₁-C₆ alkyl.

33-37. (canceled)

38. The composition of claim 1, wherein the imaging moiety is a radioisotope for nuclear medicine imaging, a paramagnetic species for use in MRI imaging, an echogenic entity for use in ultrasound imaging, a fluorescent entity for use in fluorescence imaging, or a light-active entity for use in optical imaging.

39-41. (canceled)

42. The composition of claim 38 wherein the imaging moiety is ¹⁸F.

43. The composition of claim 1, wherein the imaging agent is selected from the group consisting of

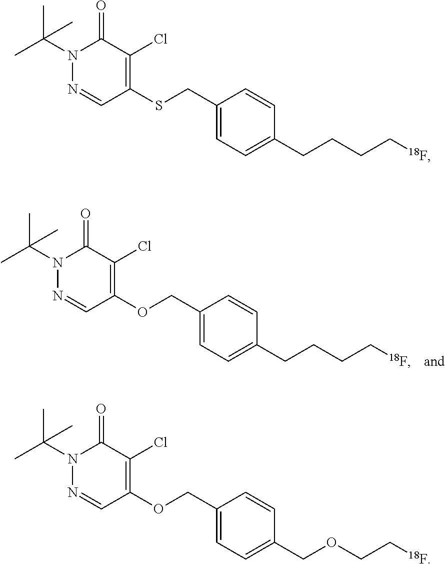

44. A method comprising administering the composition of claim 1 to a subject; and obtaining an image of the subject.

45-49. (canceled)

50. The composition of claim 1, wherein the pH of the composition is between 6.1 and 7.5.

51. The composition of claim 1, wherein the pH of the composition is between 6.6 and 7.5.
